(19) United States
(12) Patent Application Publication
(10) Pub. No.: US 2026/0240756 A1
(43) Pub. Date: Aug. 20, 2026

(54) COMPOSITION COMBINING A THERMAL WATER AND A LACTOBACILLUS ACIDOPHILUS EXTRACT AND ACTIVITY THEREOF FOR STRENGTHENING THE SKIN BARRIER AND COMBATING SKIN AGING

(71) Applicant: LABORATOIRES LEA, PERIGNY (FR)

(72) Inventor: Valérie CAUCHE, LAGORD (FR)

(73) Assignee: LABORATOIRES LEA, PERIGNY (FR)

(21) Appl. No.: 19/162,183

(22) PCT Filed: Feb. 9, 2024
(86) PCT No.: PCT/EP2024/055177

§ 371 Date: Sep. 4, 2025

(30) Foreign Application Priority Data
Mar. 6, 2023 (FR) FR2302063

(51) Int. Cl. A61K 8/99; A61K 8/19; A61Q 19/00; A61Q 19/08

(52) U.S. Cl. CPC A61K 8/99; A61K 8/19; A61Q 19/00; A61Q 19/005; A61Q 19/08

(57) ABSTRACT

Cosmetic and/or dermatological compositions have, in a physiologically acceptable medium, a thermal spring water whose mineral content is greater than 20 mg/L and a concentration of between 1% and 10% of an extract of Lactobacillus acidophilus by weight of the total weight of the composition. Methods of treatment are disclosed that include providing such a cosmetic and/or dermatological composition to a patient in need thereof. The treatments protect, reinforce, and/or repair the skin's barrier function and reduce sensations of discomfort in sensitive to reactive skin and even atopic skin. The treatment can combat skin aging and to improve the cutaneous microbiota.

### TECHNICAL FIELD

The invention relates to a cosmetic and/or dermatological composition comprising a thermal spring water combined with an extract of Lactobacillus acidophilus, and its use for skin care.

### PRIOR ART

Skin is a perpetually renewing organ that covers the surface of the body and forms the interface between the organism and the external environment. Its purpose is to protect the body from external harms, but also to combat dehydration by limiting the diffusion of water.

The skin is thus a vital organ composed of several layers (dermis, proliferative layers, and stratum corneum), which perform protective, sensitive, immune, metabolic or thermoregulatory functions. The skin, like other organs, is subject to aging.

The skin's appearance can be modified by internal alterations (intrinsic aging, disease and hormonal changes such as pregnancy) or external factors (environmental factors such as pollution, sunlight, pathogens, temperature variations, etc.). These changes may result in the appearance of wrinkles and fine lines, pigmentation defects, dryness or even dehydration of the skin, thinning of the epidermis, elastosis, blemishes, age spots, etc. Each of these changes affects not only the skin, but also keratinized appendages such as nails and hair.

Although the entire structure of the skin plays an active role in the body's defense, this cutaneous barrier is largely provided by the epidermis.

The epidermis is an epithelium subdivided into a plurality of layers or strata, from the basal layer just above the dermis, through the granular and spinous layers, to the top layer, the stratum corneum.

The role of the epidermis is essential in preventing the loss of water and other body components to the external environment, and in protecting the body against a variety of environmental harms. Its main function is to protect the body from external threats by establishing physical, chemical, biochemical, and immunological barriers to them, while maintaining a certain capacity for exchange between the external and internal environments.

The highly compact structure of the stratum corneum forms a barrier to external causes of damage, making the skin impermeable. Lipids in the intercellular cement (polyunsaturated fatty acids, cholesterol, ceramides) and keratin ensure watertightness and slow the evaporation of water from deeper layers. For this effect to be optimal, the stratum corneum must be perfectly intact: if it is hyperhydrated (under an occlusive patch, for example), the skin becomes permeable in both directions; if it is dry, the barrier cracks and becomes vulnerable.

The skin's state of hydration is the result of a balance between water diffusion and evaporation. The margin for optimal hydration is very narrow: plus or minus 2% around the average value of 13%. Skin is considered dry as soon as its moisture content falls below 10%.

If water evaporation is permanent, it is constantly renewed by diffusion mechanisms. These losses are compensated by nutritional and cutaneous water intake: water from the bloodstream supplements the water reserve in the dermis, which continues to diffuse into the upper layers of the skin.

Moisturizing the skin is therefore essential to its barrier function. The skin's water can be found in different states: non-mobilizable water, bound to biological molecules, and mobilizable water, which can pass through the different layers of the epidermis.

The dermis is the skin's water reservoir, containing about 80% of its water. It can store water because it is largely made up of a gel of proteoglycans (Extra Cellular Matrix), which encloses its constituent proteins (collagen, elastin, etc.). This gel is notably composed of hyaluronic acid, which has the ability to bind water (up to 1000 times its weight in water), retaining most of it in the dermis. This large water reserve ensures the skin's “tautness”.

A small fraction of this water is referred to as “mobilizable water”. It is able to penetrate the upper layers of the epidermis by passive diffusion. It also moisturizes corneocytes, which in addition to being rich in keratin, contain their own natural moisturizing factor (NMF), made up of several molecules such as urea, amino acids derived from the degradation of filaggrin, lactic acids, sugars and mineral salts, with water-retaining properties.

NMF accounts for up to 20-30% of the dry matter in the stratum corneum, helping to keep it moisturized, preventing the skin from feeling dry and keeping it supple.

Skin dryness increases with age due to a reduction in NMF. Similarly, any mechanical or chemical alteration of corneocytes causes NMF to leak outside the cell, reducing their ability to retain water. The level of NMF components is reduced after the skin is washed with soap. A reduction in NMF dries out the skin and disrupts its barrier function. With less protection, the skin becomes much more susceptible to damage from irritants. The NMF is therefore considered an essential component in the regulation of epidermal homeostasis, and there is a need to reinforce it.

NMF keeps the stratum corneum hydrated and the skin supple.

When the barrier function is disrupted, the epidermis triggers different signaling pathways to restore the skin's normal state. Lipid secretion is stimulated and filaggrin is degraded to NMF. These two actions take place simultaneously to restore the barrier function. Consequently, a decrease in filaggrin reveals an alteration in the skin barrier.

The main function of this protein is to form the skin barrier. It is responsible for the aggregation and compacting of intermediate keratin filaments, playing a role in the flattening of cells (corneocytes). Bundles of intermediate keratin filaments aggregated by filaggrin bind to structural proteins through the action of transglutaminases.

Filaggrin is thought to be one of the so-called “youth proteins”, like collagen and elastin.

In the stratum corneum, its role is twofold: it both helps retain water in the skin, and helps protect the epidermis from the sun's rays.

It is a marker of improved epidermal or keratinocyte differentiation. Moreover, since lipids are synthesized in keratinocytes during this epidermal differentiation, filaggrin is also involved in the metabolism and organization of skin lipids (lipid bilayer).

According to the literature, 27.5% of Caucasian Americans, 48% of Europeans, 31.4% of Chinese and 20% of Japanese with atopic dermatitis have mutations in the filaggrin gene. Some fifty loss-of-function mutations have been identified.

Nearly half of people with the most severe forms of eczema are thought to have filaggrin deficiency, and patients with genetic mutations in the gene are three times more likely to develop atopic dermatitis.

In fact, a mutation in the filaggrin gene can interfere with the skin's ability to act as a barrier, making it more prone to allergies and infections.

Furthermore, filaggrin deficiency in atopic dermatitis is linked to increased colonization by Staphylococcus aureus.

Filaggrin is therefore essential to the formation and function of the skin barrier. Mutations in the filaggrin gene are responsible for skin disorders and increase the risk of developing pathologies such as atopic dermatitis, characterized by chronic eczema, severe pruritus and skin dryness (xerosis).

Collagen production capacity also declines with age. Our skin loses an average of 1% every year, from the age of 30 onwards. As a result, the skin becomes fragile, withers and loses its firmness.

However, it is known that repeated use of certain topical medications (dermocorticoids, retinoids, etc.) can dry out the skin, which, once weakened, cannot retain water properly.

Pathological factors such as alterations to the skin barrier (dermatitis, burns, etc.) also accelerate water loss.

Finally, when water intake is insufficient, skin dehydration occurs.

Water is particularly important for the skin. It is therefore an essential ingredient in cosmetics, both as a solvent and as a moisturizer.

There are two main ways to increase skin hydration:
Reducing water evaporation, and
Increasing water binding.

When a cosmetic product is formulated, it must maintain the integrity of the skin while being pleasant to use for the consumer (texture, smell, color, etc.). It must also respect the skin's pH.

Hydration can easily be restored by temporarily using a richer dermocosmetic care product that meets the skin's needs.

Several types of substances are used in formulations:
hydrophobic film-forming agents, which remain on the skin surface after the aqueous phase of the emulsion evaporates. They form an impermeable barrier that is occlusive to some degree and reduces intrinsic water evaporation. Illustrative examples include vaseline, paraffin, beeswax, lanolin, cetyl and stearyl alcohol, fatty alcohols (caprylyl glycol, behenyl alcohol, cetearyl alcohol, octyldodecanol), silicones (cyclohexasiloxane, dimethicone), squalane, synthetic fatty esters (cetearyl isononanoate) as well as fatty acids (stearic and palmitic acid, lauroyl lysine), triglycerides (apricot oil, sweet almond oil, soybean oil) and shea butter;
intercellular cement correctors, which are incorporated into the intercorneocyte spaces to restore cohesion between cells and limit water diffusion. Illustrative examples include ceramides, phospholipids, polyunsaturated fatty acids: γ-linolenic acid (borage oil, camelina oil), linoleic acid (safflower oil, evening primrose oil);
hydrophilic film-forming agents, which have a high capacity to bind water within the stratum corneum. They are like “gels” that retain water on the skin's surface, enhancing the moisturizing capacities of other associated active ingredients. Illustrative examples include glycosaminoglycans (hyaluronic acid), collagen, chitosan, galactomannans, polyvinyl alcohol, etc.;
hygroscopic “moisturizing” substances that capture water from the environment and deliver it to the skin. They are widely used and need to be combined with lipophilic agents. Illustrative examples include glycerine, glycerol (stearate), sorbitol and propylene glycol.

These substances can be used to form emulsions. Their aqueous phase is essentially composed of water, the first element to appear in the composition of dermocosmetics. In general, demineralized, decontaminated water is used as the solvent. Substituting thermal spring water allows us to benefit from its unique physical and chemical composition, making it an active ingredient in its own right rather than a mere solvent. Its use, however, requires complex formulations with a lipophilic phase to be effective, as the minerals need to be in contact with the skin, which is a hydrophobic surface.

However, the presence of surfactants and emulsifiers (such as polyethylene glycol, polysorbate, potassium alkyl phosphate, etc.) is essential to ensure phase miscibility and emulsion stability. Various texturing agents are also incorporated.

Furthermore, to provide a biological solution for protecting the skin's barrier function and repairing it following external damage, patent document EP2986347 discloses a dermatological composition for topical use, comprising the combination of a culture supernatant and a cell lysate, said supernatant and lysate being derived from a culture of Lactobacillus pentosus (and more particularly the strain Lactobacillus pentosus CNCM 1-4730 as deposited on Apr. 4, 2013 in accordance with the Budapest Treaty at the Collection Nationale de Culture de Microorganismes (CNCM)), for use in the treatment of irritated or inflammatory skin, said supernatant and said cell lysate coming from a Lactobacillus pentosus culture in stationary phase, after centrifugation of the culture medium to obtain a supernatant and a biomass, separation of the supernatant and biomass, complete cell lysis of the biomass to obtain the lysate, and mixing of said supernatant and said lysate in a weight ratio of supernatant to lysate of 1 to 50, said combination being present in a concentration of 0.1 to 10% by weight relative to the total weight of the composition.

The authors have demonstrated that, in order to possess such properties, said culture extract must combine both a supernatant and a cell lysate from the culture, this combination making it possible to bring together proteins, peptides, polysaccharides and short-chain amino and organic acids and, more generally, all the compounds forming the bacterial cell and the metabolites produced by L. pentosus, which, in combination, improve the barrier function of the stratum corneum or accelerate its recovery when it is impaired, by nourishing the NMF.

Also known is document WO2022/013897, which discloses a dermatological composition comprising 0.1-1% (w/w) of a mixture of lysates from the fermentation of Lactobacillus acidophilus, Lactobacillus casei, Lactobacillus plantarum and a lysate of Streptococcus thermophilus. It's a dermatological composition between skincare and colored make-up base for those who want a “hybrid” solution halfway between skincare and make-up to rebalance the skin, increase skin hydration and elasticity, and at the same time, reduce skin discoloration.

A Bifidobacterium longum lysate has also been shown to benefit skin sensitivity by inhibiting the release of neurotransmitters such as substance P and improving skin barrier function. Other examples of lactic acid bacteria extracts include Streptococcus salivarius, which increased ceramide levels in the skin (Bifidobacterium longum lysate, a new ingredient for reactive skin. Audrey Guéniche et al., L'Oreal Research, 2009).

Lactobacillus ferments are also already used in cosmetics, but mainly as skin care agents and antimicrobial preservatives.

Document CN106860284 describes an anti-wrinkle composition consisting of the following numerous raw materials taken in combination for their notably antioxidant effects, in percent by weight: 0.5% ginseng extract, 0.6% arbutin, 0.3% glabridin, 0.2% snail protein powder, 0.3% licorice flavonoid liquid, 0.5% tranmetracycline acid, 0.6% aloe vera extract, 0.8% yeast extract, 0.7% honeysuckle extract, 1.3% cyanocobalamin, 1.2% cellulase, 1.5% Lactobacillus acidophilus, 1.2% euphorbia extract, 1.5% Salvia miltiorrhiza extract, moxa leaf extract, 1.6% atractylodes extract, 1.3% Gynostemma pentaphylla extract, 1.5% loofah extract, 1.7% gentian extract, 1.1% clove extract, 1.2% licorice root extract, 0.8% vitamin C, 0.9% chamomile extract, 1% Centella asiatica extract, 0.9% angelica extract, 1% lily extract, 1.1% cornflower extract, 1.5% caprylic acid triglyceride, 1.2% frankincense and 71.4% ionized water.

Leucidal® Liquid SF is known, for example, which is a probiotic derived from the fermentation of Lactobacillus acidophilus, known to moisturize the skin by reducing moisture loss.

Thermal spring waters are also known for their beneficial effects on the skin. These waters, naturally rich in minerals and trace elements, have moisturizing, soothing, remineralizing, anti-free radical, anti-inflammatory, healing, anti-irritant and decongestant properties, making them ideal for resolving many skin disorders (dryness, discomfort, irritation, etc.).

Nevertheless, no two thermal spring waters are alike. Indeed, depending on the source from which it is drawn, a thermal spring water will not have the same composition as a thermal spring water drawn from another source.

By way of example, Jonzac thermal spring water is known and used for its regenerating (reinforces the skin barrier), anti-inflammatory (limits sensitivity and reactions linked to inflammation), soothing (calms the skin and limits redness) and moisturizing (reinforces the skin's natural hydration) properties.

### Technical Problem

In view of the above, one problem which the invention sets out to solve is to provide a new biological solution for protecting the skin's barrier function and repairing it, for example following external damage.

Unexpectedly, the Applicant has demonstrated the synergistic effects of a thermal spring water and a postbiotic, enabling the degradation of filaggrin to be totally reduced, thus preserving the skin's barrier function, and also inducing an increase in Type I collagen expression. In addition, this protective or restorative action halves the proliferative response and demonstrates good anti-aging activity.

### Technical Solution

The first object of the solution to this stated problem is a cosmetic and/or dermatological composition, characterized in that it comprises, in a physiologically acceptable medium, a thermal spring water whose mineral content is greater than 20 mg/L and a concentration of between 1% and 10% of an extract of Lactobacillus acidophilus by weight of the total weight of the composition.

It also covers the uses of the composition according to the invention to protect, reinforce and/or repair the skin's barrier function, to combat skin aging and to improve the skin's microbiota.

The invention and its advantages will be better understood on reading the following description and non-limiting embodiments.

### BRIEF DESCRIPTION OF THE DRAWINGS

The invention and its advantages will be better understood on reading the following description and non-limiting embodiments, with reference to the appended figures, wherein:

FIG. 1 shows the epidermal hyperproliferation after stripping at D8 by measuring epidermal thickness on the different batches of human skin explants tested in Example 1.

FIG. 2 shows the effect of the products tested in Example 1 on the inhibition of stripping-induced epidermal hyperproliferation at D8.

FIG. 3 shows the staining of filaggrin in the stratum corneum, in the different batches of human skin explants tested in Example 1.

FIG. 4 shows the percentage of surface area occupied by filaggrin in the stratum corneum, in the different batches of human skin explants tested in Example 1.

FIG. 5 shows the staining of Type I collagen in the papillary dermis, in the different batches of human skin explants tested in Example 1.

FIG. 6 shows the percentage of surface area occupied by Type I collagen in the papillary dermis, in the different batches of human skin explants tested in Example 1.

FIG. 7 shows a table summarizing the results obtained in terms of cell viability, epidermal thickness, filaggrin expression and Type I collagen expression in the different batches of human skin explants tested in Example 1.

FIG. 8 shows a table summarizing the comparative expression values of filaggrin and Type I collagen in the different batches of human skin explants tested in Example 1.

FIG. 9 shows a linearized plot of the percentage (%) reduction in DPPH as a function of trolox concentration (mg/L).

FIG. 10 shows the free radical scavenging activity in percentage (%) DPPH reduction of a combination of 2% Lactobacillus acidophilus extract+0.5% distilled water and a combination of 2% Lactobacillus acidophilus extract+0.5% Jonzac thermal spring water.

### DESCRIPTION OF EMBODIMENTS

The invention relates to a cosmetic and/or dermatological composition, characterized in that it comprises, in a physiologically acceptable medium, a thermal spring water whose mineral content is greater than 20 mg/L and a concentration of between 1% and 10% of an extract of Lactobacillus acidophilus by weight of the total weight of the composition.

A physiologically acceptable medium denotes a medium suitable for use in contact with human and animal cells, in particular with cells of the epidermis, without toxicity, irritation, inappropriate allergic response or the like, and proportionate to a reasonable benefit-risk ratio.

Such a physiologically acceptable medium may comprise excipients known and used in the cosmetic and dermatological fields. A person skilled in the art will take care to choose the physiologically acceptable medium in such a way that they do not impair the attractive properties of the compositions according to the invention.

By thermal spring water is meant naturally mineralized spring water with a mineral content greater than 20 mg/L, preferentially greater than 150 mg/L, more preferentially greater than 1000 mg/L, even more preferentially greater than 5000 mg/L, and even more preferentially greater than 7000 mg/L.

Preferably, the thermal spring water is chosen from the thermal spring water of Jonzac, Rochefort, Avène, La Roche Posay, Uriage, Saint Gervais, Gamarde, Fumades, Cauteret, Evaux, Vichy, more preferentially the thermal spring water of Jonzac and Rochefort, even more preferentially the thermal spring water of Jonzac.

The thermal water preferentially used is a polymetallic sulfated calcic mixed thermal water, sodium chloride, magnesium, hypermineralized with a mineral content of at least 5000 mg/L, for example of the order of 7000 mg/L, essentially comprising calcium and magnesium, as well as silicon, strontium, boron, lithium and manganese, hypotonic, practically isotonic.

Preferably, the thermal spring water used is Jonzac thermal spring water.

Jonzac thermal spring water is more specifically a sulfide, mixed sulfate, sodium chloride, trace element-rich and hypermineralized type of water with a mineral content of over 6500 mg/L, for example of the order of 7000 mg/L. Among the minerals, we mainly find calcium (Ca2+), magnesium (Mg2+) and strontium (Sr).

Jonzac thermal spring water is hyperthermal (emergence temperature 62° C.), bacteriologically pure and isotonic, with a pH of 6.95.

The composition according to the invention preferentially comprises thermal spring water at a concentration of between 0.5% and 99.5% by weight of the total weight of the composition, for example 10%, 15%, 20%, 25%, 30%, 35%, 40%, 45%, 50%, 55%, 60%, 65%, 70%, 75%, 80%, 85%, 90%, 95%, preferentially between 5% and 50%, more preferentially between 10% and 35%, even more preferentially 20% or 30%.

The composition according to the invention comprises a postbiotic, that is, a preparation of inanimate micro-organisms and/or their components capable of conferring in particular a health benefit.

A postbiotic differs from a probiotic in that the latter is inactivated following the fermentation process. Thus, postbiotics may correspond to the microbial cell in its entirety, or only to certain components or metabolites.

The composition according to the invention comprises a skin postbiotic, namely an extract of Lactobacillus acidophilus.

Lactobacillus acidophilus is a Gram+, non-spore-forming, rod-shaped lactic acid bacterium with rounded ends. It is non-mobile and appears singly, in pairs or in short chains.

It is a bacterium that grows at an optimum temperature of between 35° C. and 40° C., although it is capable of growth up to 45° C. Its optimum pH for growth is between 5.5 and 6.

Most strains are aerotolerant, but growth remains optimal under microaerobic or even anaerobic conditions.

The preferentially-used Lactobacillus acidophilus extract is a Lactobacillus acidophilus cell lysate derived from the fermentation of Lactobacillus acidophilus.

According to an advantageous embodiment, the Lactobacillus acidophilus cell lysate is obtained by fermenting Lactobacillus acidophilus in a culture medium comprising ammonium sulfate, magnesium sulfate, disodium phosphate (mineral) and a natural yeast autolysate, followed by controlled lysis in the presence of a lysozyme derived from papaya (Carica papaya) and, after filtration, removal of the biomass and recovery of said cell lysate.

The Lactobacillus acidophilus extract used is, for example, an extract of the strain Lactobacillus acidophilus ATCC 314.

The composition according to the invention preferentially comprises Lactobacillus acidophilus extract at a concentration of between more than 1.5% and 10% by weight of the total weight of the composition, more preferentially between 2% and 10% by weight of the total weight of the composition, for example 2%, 3%, 4%, 5%, 6%, 7%, 8%, 9%, still more preferentially between 2% and 5%, even more preferentially 2%.

Preferentially, the composition according to the invention further comprises hyaluronic acid and its derivatives, an hydroxyproline and its derivatives such as dipalmitoyl hydroxyproline, hydrolyzed lupin protein, an algal extract such as an aqueous extract of protein-rich brown algae such as fucus and kelp, an oily extract of green algae such as alaria rich in omega 3, 6 and 9 or chlorella, or an extract of red algae such as Jania rubens, oligosaccharides (prebiotics), zinc PCA, mallow or chamomile floral water, witch hazel extract, Centella asiatica extract, aloe vera extract/juice, ceramides such as N-oleoyl-phytosphingosine (or ceramide NP), phytosphingosine, sodium carrageenan, dextrin, phytic acid, tocopherol, buckwheat wax, shea butter, castor oil, vegetable squalane, glycerine, alone or in combination.

More preferentially, the composition according to the invention also comprises hyaluronic acid and its derivatives, mallow floral water, aloe vera extract/juice, Centella asiatica extract, red algae extract such as Jania rubens, sodium carrageenan, phytosphingosine, dextrin, phytic acid, tocopherol, buckwheat wax, shea butter, castor oil, vegetable squalane, glycerine, alone or in combination.

Preferentially, the compositions according to the invention are in a form suitable for topical administration.

Illustrative examples of topical formulations according to the invention include a cream, optionally tinted, an oil-in-water (O/W) emulsion, a water-in-oil (W/O) emulsion, a gel, a gel-cream, a water, a body or lip balm, an ointment, a serum, an eye and lip contour product, a micellar water, a mask or a cleansing milk.

Another object of the invention concerns a composition according to the invention, used to protect, reinforce and/or repair the skin's barrier function and reduce sensations of discomfort in sensitive to reactive skin and even atopic skin.

Protecting, reinforcing and/or repairing the skin's barrier function means, for example, improving skin hydration, that is, any improvements in changes to the skin's outward appearance due to dehydration, such as dryness, tightness and soothing sensations of discomfort and reducing reactivity, so as to help prevent the onset of visible skin reactions, particularly on sensitive to reactive skin or even atopic skin.

The composition according to the invention is also used to combat skin aging, particularly for normal to sensitive skin, by reinforcing skin firmness.

Signs of skin aging include all age-related changes in the skin's external appearance, such as fine lines and wrinkles, cracks, bags under the eyes, dark circles, crepiness, loss of elasticity, firmness and/or tone of the skin, but also all internal changes to the skin which do not necessarily produce visible changes, such as thinning of the skin, or all internal damage to the skin.

The composition according to the invention is also used to improve skin microbiota.

By way of illustrative examples, the composition according to the invention can help make the skin less susceptible to the penetration of allergens, bacteria, viruses and inflammations and thus prevent the development of skin pathologies such as Atopic Dermatitis, prevent premature aging and the appearance of wrinkles and improve skin hydration.

### EXAMPLES

The present invention will now be shown by means of the following examples:

### Example 1: Evaluation of the Activity of Jonzac Thermal Spring Water, Lactobacillus acidophilus and their Combination on Human Skin Explants

The aim of this two-phase study is to evaluate the anti-aging and epidermal barrier-enhancing actions of two products, and their synergy on ex vivo human skin explants.

The first ex vivo phase reproduces an application of the product on the skin.

The second histological phase enables the evolution of biological parameters to be assessed by staining and immunolabeling.

The activity is assessed by:
assessment of cell viability after Masson tri-chrome staining, with measurement of epidermal thickness,
immunostaining for filaggrin, and
immunostaining for Type I collagen.

The following products were tested:
P1=Jonzac thermal spring water; and
P2=Lactobacillus acidophilus extract.

In the following examples, it is understood that P1 refers to Jonzac thermal spring water and P2 to Lactobacillus acidophilus extract.

At D0, products P1 and P2 are diluted in sterile distilled water as follows:
Batch P1=P1 at 20%;
Batch P2=P2 at 2%; and
Batch P3=P1 at 20%+P2 at 2%.

Prepared products were stored at 4° C. during the ex vivo phase of the study.

### Phase 1: Preparation of Explants

For the ex vivo phase of the study, 18 human skin explants measuring 12±1 mm in diameter, 12 of which had undergone 10 strippings (Batch S and S′x′), were prepared from an abdominoplasty of a phototype II Caucasian woman aged 69 (reference P2660-AB69).

Stripping is a model of mechanical skin irritation which involves progressively peeling away the superficial part of the epidermis, the stratum corneum. Skin irritation is achieved by successively applying and removing an adhesive strip over the same area of skin.

Explants were grown in an adapted medium at 37° C. in a humid atmosphere enriched with 5% CO2.

### Damage to the Epidermal Barrier

At D0, just before product application, explants from batches S and S′x′ were stripped 10 times with adhesive tape.

### Explant Distribution

The explants were divided into 6 batches as follows:

TABLE 1

| Batch | Designation/Treatment | Number of explants | Stop |
|---|---|---|---|
| T0 | Surgery-sourced control | 3 | D 0 |
| T | Untreated control | 3 | D 8 |
| S | Stripped control: | 3 | D 8 |
| | x10 strippings | | |
| SP1 | Strippings x10 + 20% P1 | 3 | D 8 |
| SP2 | Strippings x10 + 2% P2 | 3 | D 8 |
| SP3 | Strippings x10 + Mix of | 3 | D 8 |
| | 20% P1 + 2% P2 | | |

### Applying the Products

At D0, D2, D3 and D6, test products P1, P2 and P3 were applied topically, at a rate of 2 μL per 1 cm² explant, that is, around 2 mg/cm², and spread using a small spatula.

Explants from control batches received no treatment other than having the medium renewed.

Half of the medium (1 mL/well) was renewed at D2, D3 and D6.

### Harvesting

At D0, the 3 explants from batch TO were harvested. Each sample was cut in half, one half was frozen at −80° C. at optimal cutting temperature (OCT), the other half was fixed in formalin.

At D8, 3 explants from each batch were harvested and processed in the same way as at DO described above.

### Phase 2: Histological Treatments

The samples described above were conditioned for 24 hours in buffered formalin, then dehydrated and impregnated with paraffin using a Leica PEARL automatic dehydrator. A Leica EG 1160 embedding station was used to embed them.

Sections of 5 μm were cut using a Minot microtome, Leica RM 2125, and mounted on Superfrost® histological glass slides.

Microscopic observations were made under light microscopy, using a Leica DMLB, Olympus BX43 or Olympus BX63 microscope.

The images were captured using an Olympus DP72 or DP74 camera and cellSens software.

### 1. Cellular Viability

Cell viability of epidermal and dermal structures was assessed on paraffin sections after staining with Goldner's variant of Masson trichrome. It was assessed by microscopic examination. All batches were subjected to this handling.

### 2. Epidermis Thickness Measurement

On each image from Masson's trichrome staining, epidermal thickness was measured at several points using the Olympus CellSens software measurement module.

3 thickness measurements were made for each image, corresponding to 27 measurements per batch for all batches. All batches were subjected to this handling.

### 3. Immunostaining for Filaggrin

Filaggrin immunostaining was performed on paraffin sections with an anti-filaggrin monoclonal antibody (Santa Cruz, sc-66192, clone AKH1) diluted 1:100 in 0.3% PBS-BSA, for 1 h at room temperature, and revealed with an AlexaFluor AF488 (Life Technologies, ref. A11008). The nuclei were counterstained with propidium iodide. Immunostaining was assessed by microscopic observation. All batches were subjected to this handling.

### 4. Immunostaining for Type I Collagen

Type I Collagen was labeled on frozen sections with a polyclonal anti-Type I collagen antibody (Abcam, ab138492-1001) diluted 1:800 in PBS-BSA 0.3%, for 1 night at room temperature, and revealed in AlexaFluor AF488 (Lifetechnologies, ref. A11008). The nuclei were counterstained with propidium iodide.

Immunostaining was assessed by microscopic observation and semi-quantified by image analysis. All batches were subjected to this handling.

### Results

### 1. Cellular Viability

The cell viability of all batches is shown in the table below:

TABLE 2

Cellular viability

| Batch | Epidermis | Dermis |
|---|---|---|
| T0 | B | B |
| TJ8 | AB | B |
| SJ8 | AB | B |
| SP1J8 | AB | B |
| SP2J8 | AB | B |
| SP3J8 | AB | B |

Morphology legend:
B = Good,
AB = Fairly good,
TLA = Very slightly damaged,
LA = Slightly damaged,
MA = Moderately damaged,
ANA = Fairly clearly damaged,
NA = Clearly damaged,
TNA = Very clearly damaged

At D0, in the control batch (TO), cell viability was good in the epidermis and papillary dermis.

At D8, in the control batch (TJ8), cell viability was fairly good in the epidermis and good in the papillary dermis.

Damage to the epidermal barrier from 10 strippings did not induce any change in cell viability compared with the TJ8 control batch.

Effect of the product on cell viability, compared with batch SJ8:
Product P1 induces no change.
Product P2 induces no change.
Product P3 induces no change.

Effect of the product on cell viability, compared with batch SP1J8:
Product P3 induces no change.

Effect of the product on cell viability, compared with batch SP2J8:
Product P3 induces no change.

### 2. Measurement of Epidermal Hyperproliferation

### 2.1 Epidermis Thickness

When the skin is weakened by skin stripping, several major phenomena are observed:
an altered barrier function, reflected in a decrease in certain markers of the stratum corneum, including filaggrin and lipids;
epidermal hyper-proliferation set up by the skin to restore optimal barrier function;
increased secretion of certain pro-inflammatory cytokines, including IL-1 alpha.

When a product is tested on an ex vivo model of human skin weakened by strippings, its efficacy is assessed on several parameters, namely its ability to reduce epidermal hyper-proliferation and its activity on the expression of barrier function markers.

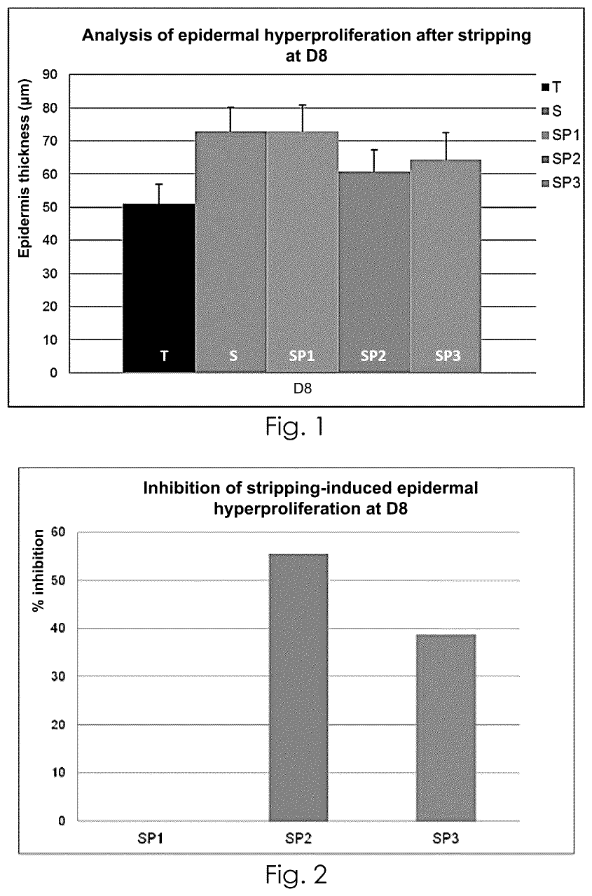

Analysis of epidermal hyperproliferation after stripping by measuring epidermal thickness on all batches is shown in the table below and shown in FIG. 1.

TABLE 3
Epidermis thickness (μm)

| | T0 | TJ8 | SJ8 | SP1J8 | SP2J8 | SP3J8 |
|---|---|---|---|---|---|---|
| Average | 27.2 | 51.1 | 72.9 | 73.0 | 60.8 | 64.5 |
| Standard deviation | 3.5 | 5.8 | 7.3 | 7.8 | 6.5 | 7.9 |

At D0, on the T0 control batch, the epidermis measures an average of 27.2 μm.

At D8, on the TJ8 control batch, the epidermis measures an average of 51.1 μm.

Damaging the epidermal barrier through 10 strippings induces epidermal hyper-proliferation, resulting in a significant 43%** increase in epidermal thickness compared to the TJ8 control batch.

Effect of product application on epidermis thickness, compared with the SJ8 control batch:
Product P1 induces no change;
Product P2 induces a significant 17%** decrease in stripping-induced epidermal hyperproliferation;
Product P3 induces a significant 12%** decrease in stripping-induced epidermal hyperproliferation.

Effect of product application on epidermis thickness, compared with the SP1J8 control batch:
Product P3 induces a significant 12%** decrease in stripping-induced epidermal hyperproliferation.

Effect of product application on epidermis thickness, compared with the SP2J8 control batch:
Product P3 induces a significant increase of 6% #.

### Caption:

Not significant: ns;
Significant: #for p<0.1 (90%); * for p<0.05 (95%); ** for p<0.01 (99%).

By stripping away the upper part of the stratum corneum, the skin's barrier function has been altered.

Following stripping (damage), the epidermis will accelerate the terminal differentiation of keratinocytes into corneocytes to reform stratum corneum. To compensate for this loss of keratinocytes, there is an activation of proliferation in the basal layer of the epidermis (see increased thickness of the stripped control epidermis).
Product P1 (ETJ 20%) does not modify the proliferative response (epidermal thickness) following loss of barrier function.
Product P2 (2% Lactobacillus acidophilus extract) halves the proliferative response. This means that P2 has a protective action against this response.

The combination of the two products halves the proliferative response and therefore also has a protective effect.

### 2.2 Inhibition of Hyper-Proliferation

The percentage inhibition of stripping-induced epidermal hyper-proliferation at D8 for the batches involved is shown in the table below and shown in FIG. 2.

TABLE 4
% inhibition of epidermal hyper-proliferation

| SP1J8 | SP2J8 | SP3J8 |
|---|---|---|
| 0% | 56% | 39% |

Effect of product application on epidermis hyper-proliferation, compared with the SJ8 control batch:
Product P1 induces no change.
Product P2 induces a significant inhibition of 56%**.
Product P3 induces a significant inhibition of 39%**.

### Caption:

Not significant: ns
Significant: #for p<0.1 (90%); * for p<0.05 (95%); ** for p<0.01 (99%).

### 3. Filaggrin

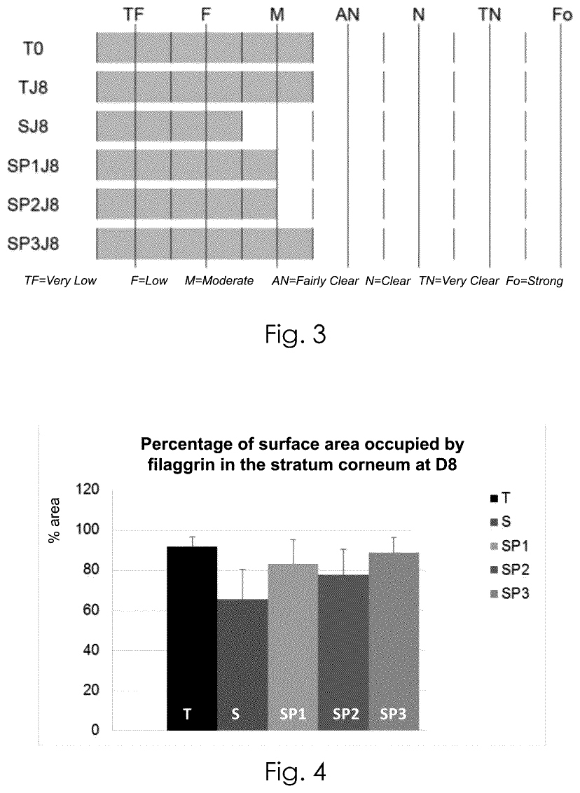

Filaggrin staining in the stratum corneum on all batches is shown in FIG. 3.

At D0, in the T0 control batch, filaggrin staining was moderate to fairly clear in the stratum corneum.

At D8, in the TJ8 control batch, filaggrin expression is moderate to fairly clear in the stratum corneum.

Damage to the epidermal barrier from 10 strippings (SJ8) induces a moderate decrease in filaggrin expression compared with the TJ8 control batch.

Effect of product application on filaggrin expression, compared with the SJ8 control batch:
Product P1 induces a slight increase;
Product P2 induces a slight increase;
Product P3 induces a moderate increase.

Effect of product application on filaggrin expression, compared with the SP1J8 control batch:
Product P3 induces a slight increase.

Effect of product application on filaggrin expression, compared with the SP2J8 control batch:
Product P3 induces a slight increase.

### Filaggrin Image Analysis

For each batch of explants, the percentage of the region of interest covered by staining (percentage of stained area) is determined by image analysis.

### Comparisons of Stained Areas

The percentage of stained area (% area) for each treatment is compared with the untreated condition.

Filaggrin image analysis was performed using cellSens software (Olympus).
Area analyzed per image: ROI including stratum corneum.
Number of images analyzed per batch: 9.
Statistical test: Student's t test.
Batches analyzed: TO, [T, S, SP1, SP2, SP3] at D8.

The percentage of surface area occupied by filaggrin in the stratum corneum is shown in the table below and shown in FIG. 4.

TABLE 5
Filaggrin (% area)

| | T0 | TJ8 | SJ8 | SP1J8 | SP2J8 | SP3J8 |
|---|---|---|---|---|---|---|
| Average | 69.3 | 91.5 | 65.4 | 83.1 | 77.4 | 88.6 |
| Standard deviation | 8.8 | 5.1 | 14.9 | 12.0 | 12.9 | 7.4 |

At D0, in the T0 control batch, filaggrin represents 69.3% of the stratum corneum area.

At D8, in the TJ8 control batch, filaggrin represents 91.5% of the stratum corneum area.

Damage to the epidermal barrier via 10 strippings induced a significant 29%** decrease in filaggrin expression compared with the TJ8 control batch.

Effect of product application on filaggrin expression, compared with the SJ8 control batch:
Product P1 induces a significant increase of 27%*;
Product P2 induces a significant increase of 18% #;
Product P3 induces a significant increase of 35%**.

Effect of product application on filaggrin expression, compared with the SP1J8 control batch:
Product P3 induces a non-significant increase of 7% ns.

Effect of product application on filaggrin expression, compared with the SP2J8 control batch:
Product P3 induces a significant increase of 14%**.

### Caption:

Not significant: ns;
Significant: #for p<0.1 (90%); * for p<0.05 (95%); ** for p<0.01 (99%).

### 4. Type I Collagen

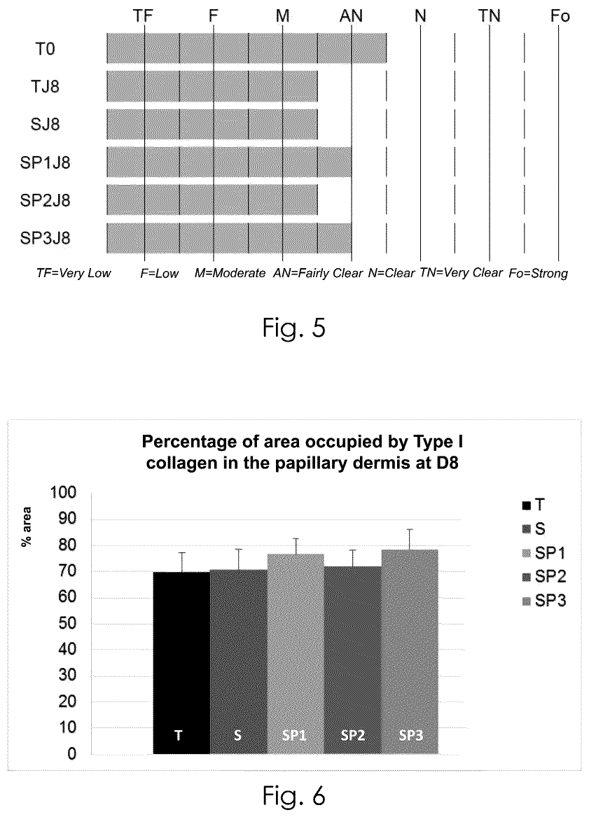

The staining of Type I collagen in the papillary dermis on all batches is shown in FIG. 5.

At D0, on the T0 control batch, Type I collagen staining is fairly clear to clear in the papillary dermis.

At D8, in the TJ8 control batch, Type I collagen expression is moderate to fairly clear in the papillary dermis.

Damage to the epidermal barrier from 10 strippings does not induce any change in Type I collagen expression in the TJ8 control batch.

Effect of product application on Type I collagen expression, compared with the SJ8 control batch:
Product P1 induces a slight increase;
Product P2 induces no change;
Product P3 induces a slight increase.

Effect of product application on Type I collagen expression, compared with the SP1J8 control batch:
Product P3 induces no change.

Effect of product application on Type I collagen expression, compared with the SP2J8 control batch:
Product P3 induces a slight increase.

### Type I Collagen Image Analysis

For each batch of explants, the percentage of the region of interest covered by staining (percentage of stained area) is determined by image analysis.

### Comparisons of Stained Areas

The percentage of stained area (% area) for each treatment is compared with the untreated condition.

Type I collagen image analysis was performed using cellSens software (Olympus).
Area analyzed per image: ROI including papillary dermis.
Number of images analyzed per batch: 9.
Statistical test: Student's t test.
Batches analyzed: T0, [T, S, SP1, SP2, SP3] at D8.

The percentage of surface area occupied by Type I collagen in the papillary dermis is shown in the table below and shown in FIG. 6.

TABLE 6
Type I collagen (% area)

| | T0 | TJ8 | SJ8 | SP1J8 | SP2J8 | SP3J8 |
|---|---|---|---|---|---|---|
| Average | 88.0 | 69.6 | 70.6 | 76.5 | 71.8 | 78.3 |
| Standard deviation | 4.0 | 7.5 | 8.0 | 6.0 | 6.3 | 7.9 |

At D8, in the TJ8 control batch, Type I collagen represents 69.6% of the papillary dermis surface.

Damage to the epidermal barrier via 10 strippings induces a non-significant 1% ns increase in Type I collagen expression compared with the TJ8 control batch.

Effect of product application on Type I collagen expression, compared with the SJ8 control batch:
Product P1 induces a significant increase of 8% #;
Product P2 induces a non-significant increase of 2% ns;
Product P3 induces a significant increase of 11% #.

Effect of product application on Type I collagen expression, compared with the SP1J8 batch:
Product P3 induces a non-significant increase of 2% ns.

Effect of product application on Type I collagen expression, compared with the SP2J8 batch:
Product P3 induces a significant increase of 9% #.

### Caption:

Not significant: ns;
Significant: #for p<0.1 (90%); * for p<0.05 (95%); ** for p<0.01 (99%).

### CONCLUSION

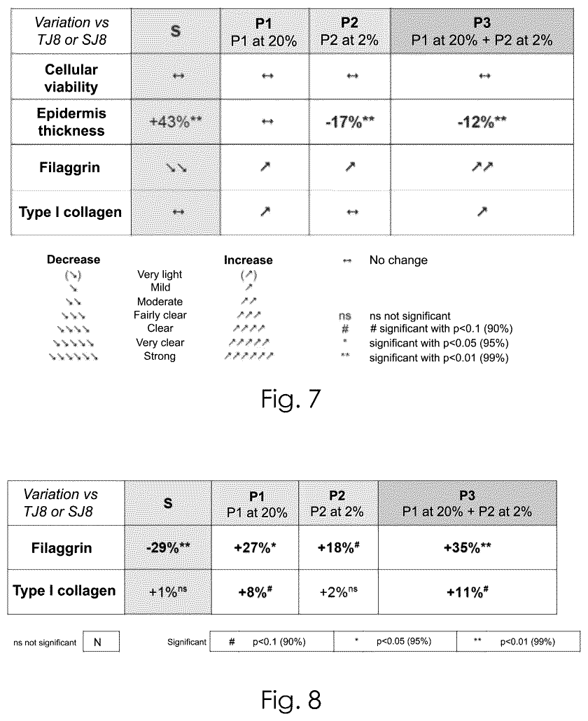

A summary of the results obtained is shown in FIGS. 7 and 8.

According to the experimental conditions described above and compared with the control batch T or the stripped batch S at D8:

After 8 days of treatment, all the products tested were well tolerated by ex vivo human skin explants.

The product “Asset 1: Jonzac Thermal Spring Water” at 20% (P1) induces a slight increase in filaggrin expression after stripping, demonstrating a skin barrier strengthening effect. It also induces a slight increase in Type I collagen expression, reflecting anti-aging activity.

The product “Asset 2: Lactobacillus acidophilus” at 2% (P2) induces a slight increase in filaggrin expression after stripping, associated with a reduction in epidermal thickness (−17%**), highlighting a good skin barrier strengthening activity.

The combination of “Asset 1: Jonzac thermal spring water” at 20% (P1) and “Asset 2: Lactobacillus acidophilus” at 2% (P2) induces an increase in filaggrin expression after stripping, associated with a reduction in epidermal thickness (−12%**), highlighting a good skin barrier strengthening activity. This combination also induces a slight increase in Type I collagen expression, reflecting good anti-aging activity.

The combination of “Asset 1: Jonzac thermal spring water” at 20% (P1) and “Asset 2: Lactobacillus acidophilus” at 2% (P2) has an additional effect on the reinforcement of the barrier effect and the expression of filaggrin and Type I collagen.

Interpretation of epidermal thickness results: by stripping away the upper part of the stratum corneum, the skin's barrier function was impaired.

In response to this loss of barrier function, filaggrin is broken down into amino acids that reinforce the NMF (Natural Moisturizing Factor) and bind large quantities of water.

At the same time the epidermis will accelerate the terminal differentiation of keratinocytes into corneocytes to reform stratum corneum. To compensate for this loss of keratinocytes, there is an activation of proliferation in the basal layer of the epidermis.

Product P1 halves the degradation of filaggrin, partially compensating for (or repairing) the skin's barrier function. Additionally, it does not modify the proliferative response (epidermal thickness) following loss of barrier function.

Product P2 halves the degradation of filaggrin, partially compensating for (or repairing) the skin's barrier function. What's more, this protective or restorative action halves the proliferative response.

The combination of the two products totally reduces the degradation of filaggrin, thus preserving the skin's barrier function. What's more, this protective or restorative action halves the proliferative response.

### Example 2: WIG Emulsion

| | % in formula |
|---|---|
| Ingredients | (w/w) |
| Emulsifier | 2-5% |
| Consistency factors | 0.5-5% |
| Emollients | 10-20% |
| Antioxidant | 0.01-0.5% |
| JONZAC thermal spring water | 15-50% |
| Organic mallow (or witch hazel) floral water | 5-25% |
| or organic aloe vera extract | 0.01-1% |
| Moisturizing agent (glycerin, propanediol, | 1-5% |
| pentylene glycol) | |
| Lactobacillus acidophilus extract | 2-10% |
| Other active ingredients (chosen from | 0.05-5% |
| phytosphingosine, dextrin, bisabolol, | |
| hyaluronic acid, buckwheat wax, Centella | |
| asiatica, Jania rubens extract, sodium | |
| carrageenan, etc.) | |
| Fragrance | 0-0.8% |
| Preservatives and anti-microbial agents | 0.001-3% |
| pH adjuster | 0.01-0.5% |
| Demineralized water | 100% qs |

### Example 3: O/W Emulsion

| | % in formula |
|---|---|
| Ingredients | (w/w) |
| Emulsifier | 2-5% |
| Co-emulsifiers | 0.1-3% |
| Consistency factors | 0.5-5% |
| Emollients | 5-25% |
| Antioxidant | 0.01-0.5% |
| JONZAC thermal spring water | 20-50% |
| Organic mallow (or witch hazel) floral water | 5-25% |
| or organic aloe vera extract | 0.01-1% |
| Moisturizing agent (glycerin, propanediol, pentylene | 1-10% |
| glycol) | |
| Gelling agent | 0.1-2% |
| Chelating agent | 0.05-0.5% |
| Lactobacillus acidophilus extract | 2-10% |
| Other active ingredients (phytosphingosine, dextrin, | 0.05-5% |
| bisabolol, hyaluronic acid, buckwheat wax, Centella | |
| asiatica, Jania rubens extract, sodium carrageenan, etc.) | |
| Fragrance | 0-0.8% |
| Preservatives and anti-microbial agents | 0.001-3% |
| pH adjuster | 0.01-0.5% |
| Demineralized water | 100% qs |

### Example 4: Micellar Water

| Ingredients | % in formula (w/w) |
|---|---|
| JONZAC thermal spring water | 15-50% |
| Organic mallow (or witch hazel) floral water | 5-25% |
| or organic aloe vera extract | 0.01-1% |
| Moisturizing agent (glycerin, propanediol, | 1-10% |
| pentylene glycol) | |
| Lactobacillus acidophilus extract | 2-10% |
| Other active ingredients (phytosphingosine, | 0.05-5% |
| dextrin, bisabolol, hyaluronic acid, buckwheat wax, | |
| Centella asiatica, Jania rubens extract, sodium | |
| carrageenan, etc.) | |
| Fragrance | 0.01-0.8% |
| Preservatives and anti-microbial agents | 0.001-3% |
| Solubilizer | 0-3% |
| pH adjuster | 0.01-0.5% |
| Demineralized water | 100% qs |

### Example 5: Care Gel/Makeup Remover

| Ingredients | % in formula (w/w) |
|---|---|
| JONZAC thermal spring water | 5-50% |
| Organic mallow (or witch hazel) floral water | 5-25% |
| or organic aloe vera extract | 0.01-1% |
| Moisturizing agent (glycerin, propanediol, | 1-15% |
| pentylene glycol) | |
| Gelling agent | 0.1-4% |
| Lactobacillus acidophilus extract | 2-10% |
| Other active ingredients (phytosphingosine, | 0.05-5% |
| dextrin, bisabolol, hyaluronic acid, buckwheat wax, | |
| Centella asiatica, Jania rubens extract, sodium | |
| carrageenan, etc.) | |
| Fragrance | 0-0.8% |
| Preservatives and anti-microbial agents | 0.001-3% |
| Solubilizer | 0-3% |
| pH adjuster | 0.01-0.5% |
| Demineralized water | 100% qs |

### Example 6: Gel-Cream

| Ingredients | % in formula (w/w) |
|---|---|
| Emulsifier | 0.5-2% |
| Consistency factors | 0.5-5% |
| Emollients | 2-25% |
| Antioxidant | 0.01-0.5% |
| JONZAC thermal spring water | 0.05-50% |
| Organic mallow (or witch hazel) floral water | 5-25% |
| or organic aloe vera extract | 0.01-1% |
| Moisturizing agent (glycerin, propanediol, | 1-10% |
| pentylene glycol) | |
| Gelling agent | 0.2-2% |
| Emulsifying gelling agent | 0.05-2% |
| Chelating agent | 0.1-0.3% |
| Lactobacillus acidophilus extract | 2-10% |
| Other active ingredients (phytosphingosine, | 0.05-5% |
| dextrin, bisabolol, hyaluronic acid, buckwheat | |
| wax, Centella asiatica, Jania rubens extract, | |
| sodium carrageenan, etc.) | |
| Fragrance | 0.05-0.8% |
| Preservatives and anti-microbial agents | 0.001-3% |
| pH adjuster | 0.01-0.5% |
| Demineralized water | 100% qs |

### Example 7: Evaluation of the Anti-Free Radical Activity of a Combination of Active Ingredients Using the DPPH (1,1-Diphenyl-2-Picrylhydrazyl) Test

The aim of this study is to evaluate the anti-free radical potential of the combination of a Lactobacillus acidophilus extract and Jonzac thermal spring water.

The anti-free radical activity of the product studied is assessed by measuring the rate of reduction of the DPPH radical in the presence of said product. The rate of reduction of the DPPH (1,1-Diphenyl-2-Picrylhydrazyl) radical is assessed by the variation in absorbance (abs) of a DPPH solution in the presence of the tested product. DPPH is a stable colored radical. Reduction of the DPPH radical by a 1-1 atom donor leads to the formation of colorless 1,1-diphenyl-2-picrylhydrazine (DPPH-H). The reaction mixture then changes from dark to light violet, or even colorless for the most strongly-antioxidant products.

The absorbance of the solutions is measured at 517 nm after 24 h incubation in the presence of the test product in a solution of DPPH (0.126 mM in ethanol).

The percentage reduction in DPPH is calculated using the following formula:

$$\% DPPH reduction = ( ( Abs reference - Abs test element ) / Abs reference ) * 100 , where : \\ Abs reference = solvent absorbance ; and \\ Abs test element = absorbance of test element .$$

The concentration of the test element giving 50% DPPH reduction is determined (IC50). It reflects its anti-free radical activity.

The activity of the test element is compared with that of a well-known anti-free radical active ingredient: Trolox.

Four products were tested:
the combination of 2% Lactobacillus acidophilus extract+0.5% distilled water;
the combination of 2% Lactobacillus acidophilus extract+0.5% Jonzac thermal spring water;
Jonzac thermal spring water alone; and
Trolox (positive control).

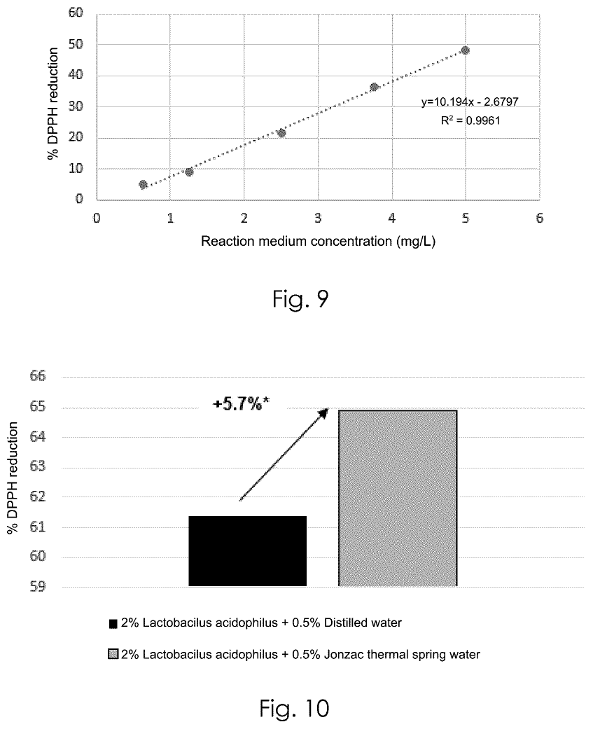

The results of the DPPH test thus obtained are presented in the table below, and the linearized graph of % DPPH reduction as a function of trolox concentration is shown in FIG. 9.

TABLE 7
DPPH test results with Trolox

| Concentration | Refer- | | | | | |
|---|---|---|---|---|---|---|
| (mg/L) | ence | 0.625 | 1.25 | 2.5 | 3.75 | 5 |
| % DPPH reduction | / | 4.969 | 9.078 | 21.548 | 36.550 | 48.256 |

The IC50 of trolox, the effective concentration corresponding to a 50% reduction in DPPH activity, is determined using the equation for the line (ax+b) shown in FIG. 9, according to the following formula: IC50 (mg/L)=(50−b)/a.

This gives the value: IC50 trolox=5.168 mg/L.

This control passes the test.

For the Jonzac thermal spring water test, the following final concentrations (% w/v) were analyzed. The results of the DPPH test are shown in the table below.

TABLE 8
DPPH test results with Jonzac thermal spring water

| Concentration | Refer- | | | | | |
|---|---|---|---|---|---|---|
| (% w/v) | ence | 2.5 | 2 | 1.5 | 1 | 0.5 |
| % DPPH | / | −2.676 | −2.793 | −1.784 | −0.698 | −1.474 |
| reduction | | | | | | |

For the test with the combination of 2% Lactobacillus acidophilus extract+0.5% Jonzac thermal spring water, the test element was tested at the following final concentrations (% w/v). The results thus obtained are shown in the table below.

Jonzac thermal spring water alone has no anti-free radical properties.

TABLE 9
DPPH test results with the combination of 2% Lactobacillus acidophilus
extract + 0.5% Jonzac thermal spring water

| | | 2% Lactobacillus acidophilus |
|---|---|---|
| Concentration | | extract + 0.5% Jonzac thermal |
| (% w/v) | Reference | spring water |
| % DPPH | / | 64.885 |
| reduction | | |

To compare antioxidant power, Jonzac thermal spring water was replaced by distilled water in the following final concentrations (% w/v). The combination of 2% Lactobacillus acidophilus extract+0.5% distilled water was tested with DPPH, and the results obtained are presented in the table below.

TABLE 10
DPPH test results with the combination of 2% Lactobacillus acidophilus
extract + 0.5% distilled water

| Concentration | | 2% Lactobacillus acidophilus |
|---|---|---|
| (% w/v) | Reference | extract + 0.5% Distilled water |
| % DPPH | / | 61.363 |
| reduction | | |

The anti-free radical power of the combination of Jonzac thermal spring water and Lactobacillus acidophilus extract is assessed by comparison with the anti-free radical power of the combination of distilled water and Lactobacillus acidophilus extract. The results are shown in FIG. 10.

A statistical test (Student's t-test) is performed to assess the significance of the difference in free radical scavenging power between the two combinations. Student's t-test gives the probability of two batches being significantly different. The difference between two batches is significant if p<0.1(#), that is, a 90% probability of two batches being significantly different, or p<0.05(*), that is, a 95% probability of two batches being significantly different, or p<0.01(**), that is, a 99% probability of two batches being significantly different.

The results obtained show that the combination of a Lactobacillus acidophilus extract with distilled water, at the concentrations tested, reveals a DPPH radical scavenging effect, with a DPPH Reduction Percentage of 61.363%, greater than 50%.

In comparison, the combination of Lactobacillus acidophilus extract with Jonzac thermal spring water (instead of distilled water), at the concentrations tested, demonstrated a DPPH radical scavenging effect, with a DPPH Reduction Percentage of 64.885%, greater than 50%.

The Student's t-test obtained gives a value of p=0.013, that is, p<0.05(*), which indicates a 95% probability that the two batches tested are significantly different.

In conclusion, under the same conditions, compared to distilled water, the addition of Jonzac thermal spring water (used at a concentration of at least 0.5%), significantly improves the antioxidant effect of Lactobacillus acidophilus extract (used at a concentration of at least 2%). There was a significant synergistic effect of +5.67% (p<0.05).

1. A cosmetic and/or dermatological composition comprising:
in a physiologically acceptable medium, a thermal spring water whose mineral content is greater than 20 mg/L and a concentration of between 1% and 10% of an extract of Lactobacillus acidophilus by weight of the total weight of the composition.

2. The composition according to claim 1, wherein the thermal spring water is chosen from the thermal spring water of Jonzac, Rochefort, Avene, La Roche Posay, Uriage, Saint Gervais, Gamarde, Fumades, Cauteret, Evaux, or Vichy.

3. The composition according to claim 1, wherein the thermal spring water is present at a concentration of between 0.5% and 99.5% by weight of the total weight of the composition.

4. The composition according to claim 1, wherein the Lactobacillus acidophilus extract is a Lactobacillus acidophilus cell lysate derived from the fermentation of Lactobacillus acidophilus.

5. The composition according to claim 1, wherein the Lactobacillus acidophilus extract is present at a concentration of between more than 1.5% and 10% by weight of the total weight of the composition.

6. The composition according to claim 1, further comprises an active ingredient selected from the group consisting of hyaluronic acid and its derivatives, hydroxyproline and its derivatives, hydrolyzed lupin protein, an algal extract, oligosaccharides, zinc PCA, mallow or chamomile floral water, witch hazel extract, Centella asiatica extract, aloe vera extract, aloe vera juice, ceramides, phytosphingosine, sodium carrageenan, dextrin, phytic acid, tocopherol, buckwheat wax, shea butter, castor oil, vegetable squalane, glycerine, and combinations thereof.

7. The composition of claim 1, wherein the composition is a topical composition.

8-10. (canceled)

11. The composition according to claim 1, wherein the thermal spring water is from Jonzac.

12. The composition according to claim 1, wherein the thermal spring water is present at a concentration of between 5% and 50%.

13. The composition according to claim 1, wherein the thermal spring water is present at a concentration of between 10% and 35%.

14. The composition according to claim 1, wherein the thermal spring water is present at a concentration of between 20% or 30%.

15. The composition according to claim 1, wherein the Lactobacillus acidophilus extract is present at a concentration of between 2% and 10%.

16. The composition according to claim 1, wherein the Lactobacillus acidophilus extract is present at a concentration of between 2% and 5%.

17. The composition according to claim 1, wherein the Lactobacillus acidophilus extract is present at a concentration of 2% by weight of the total weight of the composition.

18. A skin care method comprising:
providing a composition according to claim 1; and
administering the composition topically to skin of a user in need thereof;
wherein the composition improves skin microbiota or reduces evidence of skin aging.

19. A skin care method comprising:
providing a composition according to claim 1; and
administering the composition topically to skin of a user in need thereof;
wherein the composition protects, reinforces, and/or repairs a barrier function of the skin and reduces sensations of discomfort in sensitive to reactive skin.

20. The method of claim 19, wherein the composition reduces sensations of discomfort in atopic skin.
